(19) United States
(12) Patent Application Publication
(10) Pub. No.: US 2025/0318738 A1
(43) Pub. Date: Oct. 16, 2025

(54) PULSE WAVE DETECTING DEVICE

(71) Applicant: Taiyo Yuden Co., Ltd., Tokyo (JP)

(72) Inventors: Katsuhiro OYAMA, TOKYO (JP); Aiki KAMEYAMA, TOKYO (JP)

(21) Appl. No.: 19/248,633

(22) Filed: Jun. 25, 2025

Related U.S. Application Data
(63) Continuation of application No. PCT/JP2023/021574, filed on Jun. 9, 2023.

(30) Foreign Application Priority Data
Dec. 27, 2022 (JP) 2022-210595

(51) Int. Cl. A61B 5/021; A61B 5/00

(52) U.S. Cl. CPC A61B 5/02108; A61B 5/6824; A61B 5/681

(57) ABSTRACT

A pulse wave detecting device includes a sensor substrate, a plurality of light emitting elements, a plurality of light receiving elements, and a processing circuit. The plurality of light emitting elements are arranged in one row on a principal surface of the sensor substrate and configured to emit light. The plurality of light receiving elements are arranged in one row parallel with the row of the plurality of light emitting elements on the principal surface and configured to receive return light. The processing circuit is configured to change light outputs of the plurality of light emitting elements differently for each light emitting element based on first signals output by at least two light receiving elements of the plurality of light receiving elements, and detect a pulse wave based on second signals output by the plurality of light receiving elements after the changing of the light outputs.

### CROSS-REFERENCE TO RELATED APPLICATIONS

This application is a continuation of PCT International Application No. PCT/JP2023/021574 filed on Jun. 9, 2023, which designates the United States, incorporated herein by reference, and which claims the benefit of priority from Japanese Patent Application No. 2022-210595, filed on Dec. 27, 2022, incorporated herein by reference.

### FIELD

The present embodiment relates to a pulse wave detecting device.

### BACKGROUND

Recently, the development of smart watches having a function of a pulse wave detecting device has been actively under way. Such a smart watch includes a photoelectric pulse wave sensor for obtaining biological information related to a blood vessel. The photoelectric pulse wave sensor represents a method of measuring a volume change in blood from reflected light by irradiating the blood vessel with light in a near-infrared-to-green wavelength band and utilizing a light absorbing characteristic of hemoglobin in the blood. The smart watch calculates a pulse wave from the volume change obtained from the photoelectric pulse wave sensor.

Related techniques are described in Japanese Patent No. 5327194, and Japanese Patent No. 6806052

A conventional smart watch is generally designed such that the photoelectric pulse wave sensor can be fixed at a position that a blood vessel of an arm passes when a belt is fastened.
However, according to such a fixing method, accuracy of detection of the pulse wave may be degraded by a displacement of the photoelectric pulse wave sensor from the position that the blood vessel passes, due to body motion or other causes.

In addition, a comfortable wearing feeling is impaired when the photoelectric pulse wave sensor is to be firmly fixed by tightly fastening the belt in order to prevent a degradation in the accuracy of detection of the pulse wave.

There are accordingly needs to provide a pulse wave detecting device that can detect a pulse wave with high accuracy even when a sensor is displaced from a position that a blood vessel passes.

### SUMMARY

According to an aspect of the present invention, a pulse wave detecting device includes a sensor substrate, a plurality of light emitting elements, a plurality of light receiving elements, and a processing circuit. The plurality of light emitting elements are arranged in one row on a principal surface of the sensor substrate and configured to emit light. The plurality of light receiving elements are arranged in one row parallel with the row of the plurality of light emitting elements on the principal surface and configured to receive return light. The processing circuit is configured to change light outputs of the plurality of light emitting elements differently for each light emitting element on a basis of first signals output by at least two light receiving elements of the plurality of light receiving elements, and detect a pulse wave on a basis of second signals output by the plurality of light receiving elements after the changing of the light outputs.

### BRIEF DESCRIPTION OF THE DRAWINGS

FIG. 1 is an external view of a smart watch as a pulse wave detecting device according to an embodiment when it is fitted to a human body.

FIG. 2 is a sectional view of the smart watch sectioned along a YZ plane in FIG. 1.

FIG. 3 is a diagram illustrating an example of an arrangement of a plurality of light emitting elements and a plurality of light receiving elements provided to a sensor substrate.

FIG. 4 is a diagram illustrating an example of a volume pulse wave detected by a photoelectric pulse wave sensor.

FIG. 5 is a schematic diagram illustrating positional relations of the plurality of light emitting elements and the plurality of light receiving elements to an arm when a wearer wears the smart watch.

FIG. 6 is a diagram illustrating an example of a relation between a displacement amount of the photoelectric pulse wave sensor in a circumferential direction of the arm and amplitude of the pulse wave.

FIG. 7 is a schematic diagram illustrating an example of a configuration of the photoelectric pulse wave sensor.

FIG. 8 is a schematic diagram illustrating an example of a configuration of a microcomputer unit.

FIG. 9 is a schematic diagram of assistance in explaining an example of contents of first correspondence information.

FIG. 10 is a diagram illustrating an example of contents of second correspondence information.

FIG. 11 is a flowchart illustrating an example of operation of the smart watch.

### DETAILED DESCRIPTION

A pulse wave detecting device according to an embodiment can be mounted in an electronic apparatus such as a watch or a sphygmomanometer. Here, as an example, the pulse wave detecting device is mounted in a smart watch. It is to be noted that a position to which the pulse wave detecting device according to the embodiment is fitted is not limited to an arm.

In addition, the pulse wave detecting device according to the embodiment can detect a pulse wave, and output the pulse wave or desired biological information related to a blood vessel which biological information is calculated on the basis of the pulse wave.
The biological information related to the blood vessel which biological information can be calculated on the basis of the pulse wave is a pulse, a blood pressure, a degree of progress of arteriosclerosis, or other information.
As an example, the smart watch in the following will be described as a device that detects the pulse wave and computes detection data thereof.

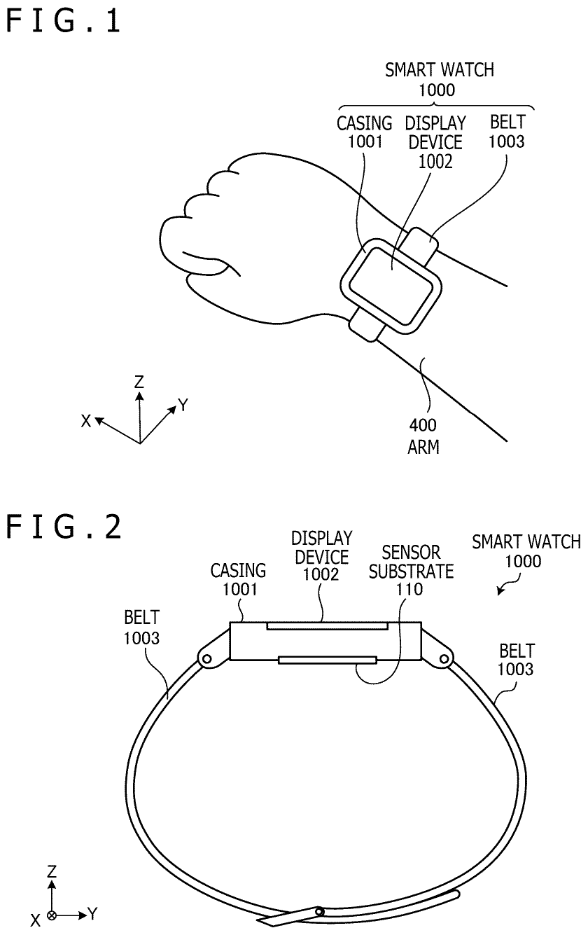

FIG. 1 is an external view of a smart watch 1000 when it is fitted to a human body in the present embodiment. FIG. 2 is a sectional view of the smart watch 1000 sectioned along a YZ plane in FIG. 1.

The smart watch 1000 includes a casing 1001 in a flat shape, a display device 1002 attached to a surface of this casing, and a belt 1003 attached to two side surfaces facing each other of the casing 1001. The casing 1001 has an upper surface, a lower surface, and side surfaces, the side surfaces connecting the periphery of the upper surface and the periphery of the lower surface to each other. In the figure, the casing 1001 is substantially formed by a rectangular parallelepiped and has four side surfaces.

The belt 1003 is attached to one side surface and another side surface of the casing 1001. The lower surface of the casing 1001 is fixed to an arm 400 of a wearer when the belt 1003 is wound around the arm 400. The upper surface of the casing 1001 is provided with the display device 1002. The smart watch 1000 outputs various kinds of biological information in the form of images to the display device 1002. The wearer can visually check the various kinds of biological information output to the display device 1002. The various images include the time and the pulse wave detected by the mounted pulse wave detecting device.

Incidentally, in the following, description will be made supposing that, as directions indicating positional relations between constituent elements of the smart watch 1000, a direction extending from the arm to a hand when the smart watch 1000 is fitted is the direction of a +X axis, a direction of going from the lower surface of the two surfaces of the casing 1001 to the upper surface is the direction of a +Z axis, and an axis orthogonal to both an X-axis and a Z-axis is a Y-axis. For example, the direction of a +Y axis is a direction of going from a thumb side to a little finger side of the arm 400.

As illustrated in FIG. 2, the lower surface of the casing 1001 is provided with a sensor substrate 110 of a photoelectric pulse wave sensor (photoelectric pulse wave sensor 100 to be described later). The lower side of the sensor substrate 110 is provided with a plurality of light emitting elements E and a plurality of light receiving elements R such that light emitting and receiving surfaces thereof face the arm.

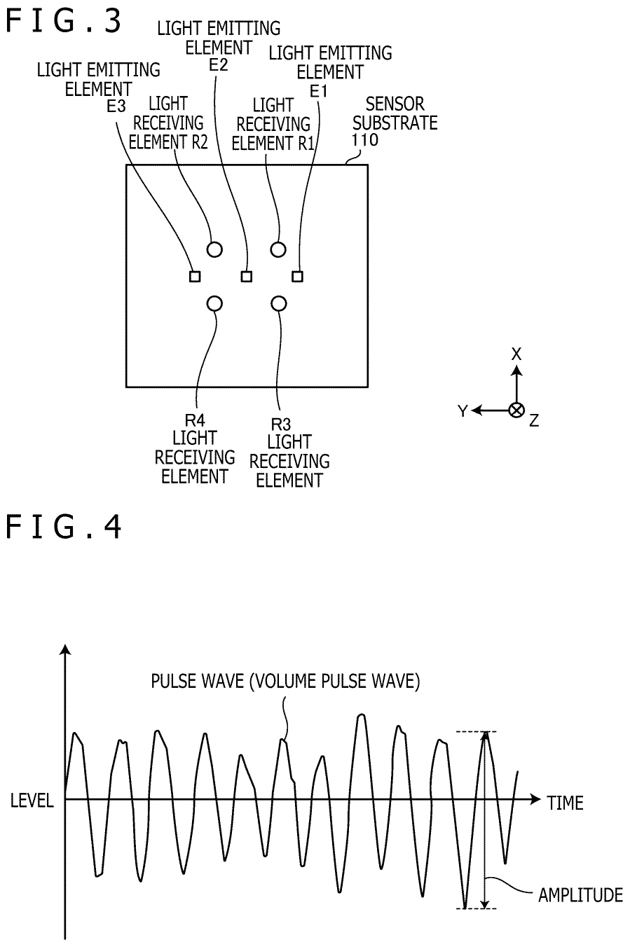

FIG. 3 is a diagram illustrating an arrangement of the plurality of light emitting elements E and the plurality of light receiving elements R provided to the sensor substrate 110.

In the example illustrated in FIG. 3, the sensor substrate 110 is of a rectangular shape.

At the center of a principal surface of the sensor substrate 110, light emitting diodes (LEDs) are used as an example of the plurality of light emitting elements E, and a light emitting element E1, a light emitting element E2, and a light emitting element E3 are arranged in one row in this order in a Y-direction. In this case, the light emitting element E2 is provided on a central point of the principal surface of the sensor substrate. Incidentally, the principal surface of the sensor substrate 110 is an abutting surface that faces the skin of the arm 400 when the smart watch 1000 is fitted to the arm 400 by the belt 1003.

In addition, in the principal surface of the sensor substrate 110, photodiodes or photosensors are used as an example of the plurality of light receiving elements R. A light receiving element R1 and a light receiving element R2 are arranged at positions separated in a +X direction from a first row of the three light emitting elements E1, E2, and E3 in such a manner as to be parallel with the first row. In addition, a light receiving element R3 and a light receiving element R4 are arranged at positions separated in a −X direction from the first row in such a manner as to be parallel with the first row.

In addition, the four light receiving elements R1 to R4 are arranged at the vertices of an imaginary rectangle. The three light emitting elements E1 to E3 and the four light receiving elements R1 to R4 have such a positional relation that the light emitting element E2 is located at the center of the rectangle and the light emitting element E1 and the light emitting element E3 are located outside the rectangle.

The light emitting elements E1 to E3 emit light selected from a wavelength range having a characteristic of being easily absorbed by hemoglobin in the blood, for example, a wavelength range from a green color to near-infrared wavelengths.
The light emitting elements E1 to E3 each emit light of the same wavelength. Each of the light emitting elements E1 to E3 is an LED.

The light receiving elements R1 to R4 can detect the light emitted by the light emitting elements E1 to E3. Each of the light receiving elements R1 to R4 is a photodiode as an example.

When the wearer wears the smart watch 1000, the light emitting elements E1 to E3 irradiate the skin of the arm 400 with the light. The light receiving elements R1 to R4 receive light incident on the light receiving elements R1 to R4 themselves including light entering under the skin and returning by being reflected or dispersed by a tissue under the skin, and output a signal corresponding to the amount of the light. This light that returns from the living body and enters the light receiving elements R1 to R4 may be referred to as return light. There are blood vessels including a radial artery, for example, under the skin of the arm 400. The amount of the return light to the light receiving elements R1 to R4 therefore increases or decreases by being affected by the volume pulse wave of the blood vessel. The photoelectric pulse wave sensor 100 provided to the smart watch 1000 detects, as the volume pulse wave, a temporal change in the amount of the light received by the light receiving elements R1 to R4.

FIG. 4 is a diagram illustrating an example of the volume pulse wave detected by the photoelectric pulse wave sensor 100. In the figure, an axis of abscissas indicates time, and an axis of ordinates indicates the level of a waveform (intensity of light).

The photoelectric pulse wave sensor 100 can obtain the waveform of the volume pulse wave that periodically increases and decreases as illustrated in FIG. 4, by performing predetermined processing such as noise removal. This waveform of the volume pulse wave will hereinafter be described simply as a pulse wave.

In a conventional smart watch, when the photoelectric pulse wave sensor is displaced from a position that the blood vessel passes due to body motion or other causes, the amount of light incident on the light receiving elements changes, and thus the amplitude of the detected pulse wave varies. Due to this phenomenon, the detection of the pulse wave with high accuracy has been difficult.
In addition, when the belt is fastened tightly to prevent this positional displacement, the detection of the pulse wave with high accuracy is possible, but wearing comfort is impaired.

According to the embodiment, the detection of the pulse wave with high accuracy is made possible even when the photoelectric pulse wave sensor 100 is displaced due to body motion or other causes. It is thus possible to maintain the accuracy of detection of the pulse wave even when the wearer fastens the belt 1003 loosely.

As a first configuration for obtaining the above-described effect, according to the embodiment, the plurality of light receiving elements R are arranged in one row on both sides of and in parallel with the first row of the plurality of light emitting elements E and at intervals equal to those of the first row.
It is thus possible to prevent a degradation in the detection accuracy caused by a displacement in a direction in which the light emitting elements E (and the light receiving elements R) are arranged.

As can be seen in FIG. 3, three light emitting elements are arranged at equal intervals in the Y-axis direction (in the first row). Incidentally, the light emitting element E2 constitutes the center of the sensor substrate 110. A far side light emitting element is E1, and a near side light emitting element is E3.
Meanwhile, a second row of the light receiving elements R1 and R2 and a third row of the light receiving elements R3 and R4 are arranged in such a manner as to be separated from the first row at equal intervals on an upper side and a lower side.
Further, concerning the imaginary rectangle formed by connecting the light receiving elements R1 to R4 to each other, a first imaginary line connecting the light receiving elements R1 and R3 to each other passes between the light emitting elements E1 and E2. In addition, a second imaginary line on a left side connecting the light receiving elements R2 and R4 to each other passes between the light emitting elements E2 and E3. Hence, the light emitting elements E1 and E3 lie off the first and second imaginary lines to a right side and a left side, respectively, by the same amount of distance.
In addition, the light emitting element E2 is provided at the center of the imaginary rectangle.

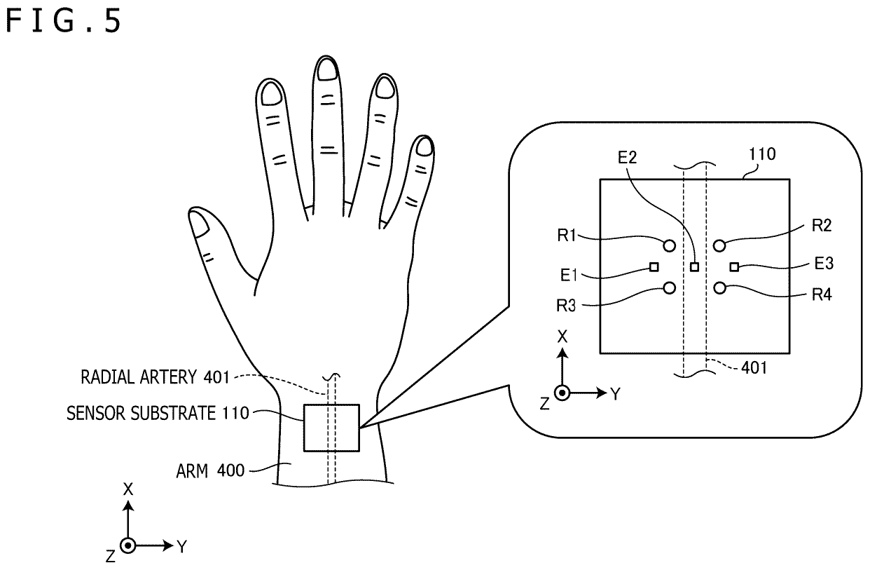

FIG. 5 is a schematic diagram illustrating positional relations of the light emitting elements E1 to E3 and the light receiving elements R1 to R4 when the smart watch 1000 is fitted to the arm.

A radial artery 401 extends in roughly the same direction as the direction in which the arm 400 extends (that is, the X-axis direction). The radial artery 401 is located between the first imaginary line and the second imaginary line.
The direction in which the radial artery 401 of the wrist extends when the smart watch 1000 is fitted roughly coincides with the X-axis direction.

Moreover, in this fitting method, in a projection view in the Z-axis direction, the radial artery 401 overlaps the light emitting element E2 disposed at the center and is sandwiched by the light emitting element E1 and the light emitting element E3. In addition, in the Y-axis direction, the radial artery 401 is sandwiched by the light receiving element R1 and the light receiving element R2 and is sandwiched by the light receiving element R3 and the light receiving element R4.

Because the light emitting elements E1 to E3 and the light receiving elements R1 to R4 are located in such relative positions with respect to the radial artery 401, the light receiving elements R1 to R4 can efficiently receive the light returning from the vicinity of the radial artery 401 after the light emitting elements E1 to E3 emit the light. Such position of the photoelectric pulse wave sensor 100 that the positional relations illustrated in FIG. 5 are obtained will be described as a normal position.

As displacement manners of the photoelectric pulse wave sensor 100, two kinds of displacement manner are possible, that is, a displacement manner in which the photoelectric pulse wave sensor 100 is displaced in the direction in which the arm 400 extends (that is, the X-axis direction) and a displacement manner in which the photoelectric pulse wave sensor 100 is displaced in a circumferential direction of the arm 400 (that is, the Y-axis direction).

As described above, the direction in which the radial artery 401 extends roughly coincides with the X-axis direction. Hence, even when the photoelectric pulse wave sensor 100 is displaced in the direction in which the arm 400 extends, the positional relations of the radial artery 401 to the light emitting elements E1 to E3 and the light receiving elements R1 to R4 hardly change. That is, even when the photoelectric pulse wave sensor 100 is displaced in the direction in which the arm 400 extends, the amplitude of the pulse wave hardly changes, and thus the accuracy of detection of the pulse wave is hardly degraded.

When the photoelectric pulse wave sensor 100 is displaced in the circumferential direction of the arm 400 (that is, the Y-axis direction), the positional relations between the radial artery 401, the light emitting elements E1 to E3, and the light receiving elements R1 to R4 change.

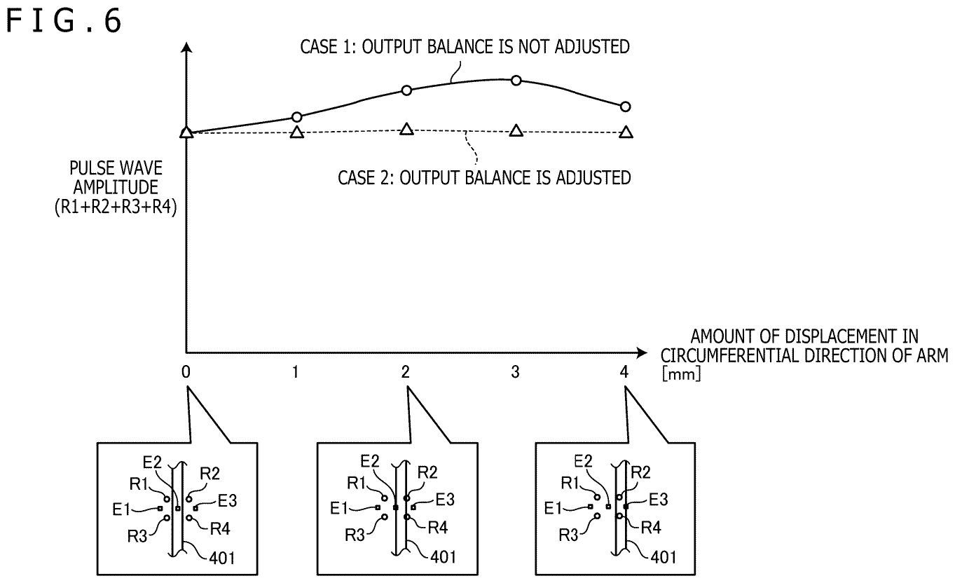

FIG. 6 is a diagram illustrating an example of a relation between the displacement amount of the photoelectric pulse wave sensor 100 in the circumferential direction of the arm 400 and the amplitude of the pulse wave. An axis of abscissas indicates the amount of displacement of the photoelectric pulse wave sensor 100 from the normal position in the circumferential direction of the arm 400. An axis of ordinates indicates the total amplitude of the pulse wave obtained from a total of output signals of the light receiving elements R1 to R4.
Incidentally, the present figure represents data obtained by performing an optical simulation supposing that the pitch of the light emitting elements E1, E2, and E3 is 5 mm, that an interval between the light receiving element R1 and the light receiving element R2 is 6 mm, and that an interval between the light receiving element R3 and the light receiving element R4 is 6 mm.

Incidentally, in the following, suppose that the wording “displacement” refers to a displacement of the photoelectric pulse wave sensor 100 in the circumferential direction of the arm 400, and that the wording “displacement amount” refers to the amount of displacement of the photoelectric pulse wave sensor 100 in the circumferential direction of the arm 400.

A curve of a case 1 represents changes in the total value of the amplitude of the pulse wave under conditions where all of light outputs of the light emitting elements E1, E2, and E3 are set to be the same output.
In the case 1, the displacement amount was changed, and when the displacement amount was 3 mm, an upwardly projecting curve was obtained with the amplitude at a peak.
With a conventional photoelectric pulse wave sensor, the outputs of the light emitting elements are decreased as they are displaced from the radial artery.
However, in contrast, it has been found that, in the case of the arrangement of FIG. 5 in the present application, the output is increased, and the detection accuracy is improved over that of the conventional sensor.
However, in the case 1, the total value of the amplitude of the pulse wave changes when displacement occurs, and therefore, the pulse wave of the radial artery is not completely accurately recognized.
In a case 2, the total value of the amplitude of the pulse wave was made substantially constant even when the displacement amount was changed. For example, a mechanism for correcting the light outputs of the respective light emitting elements E1, E2, and E3 was provided. Hence, even when a displacement occurs, the output of the pulse wave is substantially constant, and thus the detection accuracy can be improved over that of the case 1. A concrete method for this correction will be described later.
In the curve of the case 1, the light outputs of the light emitting elements E1, E2, and E3 were made to be the same. The amplitude of the pulse wave with respect to the displacement amount under this condition is illustrated. This amplitude of the pulse wave is a total of pulse wave amplitudes output by the four light receiving elements.
Also in the following description, the total of the pulse wave amplitudes will be referred to simply as the “amplitude of the pulse wave.”
For the amplitude of the pulse wave of each individual light receiving element, the photoelectric pulse wave sensor 100 was fixed at a position of a certain displacement amount, a measurement was performed in a fixed period, the amplitude of the pulse waveform in each cycle was obtained for the pulse waveform in a plurality of cycles in this period, and an average was obtained.
The amplitude of the pulse wave changes when there is a displacement amount. As indicated by the curve of the case 1, even when the photoelectric pulse wave sensor 100 was displaced from the normal position, an amplitude larger than when the displacement was 0 was obtained. Even when the photoelectric pulse wave sensor 100 was displaced by approximately 4 mm from the normal position, a larger pulse wave amplitude than when there was no displacement amount was obtained. This is considered to be attributable to reasons described in the following.

The row of the light emitting elements E1 to E3 intersects the radial artery 401 in a projection view in the Z-axis direction. Hence, it is inferred that, even when an interval between the light emitting element E2 and the radial artery 401, the light emitting element E2 being located at the center among the light emitting elements E1 to E3, is widened by a displacement of the photoelectric pulse wave sensor 100, an interval between the light emitting element E1 or E3 and the radial artery 401 is shortened at the same time.
It is understood that, even when a displacement of approximately 4 mm occurs, for example, a decrease in the amount of light according to the displacement is suppressed, and the accuracy is not decreased as compared with a conventional device.

The row of the light receiving elements R1 and R2 and the row of the light receiving elements R3 and R4 also similarly intersect the radial artery 401 in a projection view in the Z-axis direction.
Hence, even when an interval between certain light receiving elements (light receiving elements R1 and R3 in the example of FIG. 6) and the radial artery 401 is widened by a displacement of the photoelectric pulse wave sensor 100, an interval between the other light receiving elements (light receiving elements R2 and R4 in the example of FIG. 6) and the radial artery 401 is shortened.
Consequently, as for the amount of light incident on the light receiving elements R1 to R4 from the radial artery 401, even when a displacement within approximately 4 mm occurs, the light amount is not greatly decreased according to the displacement.

Thus, decreases in the amount of light reaching the radial artery 401 from the light emitting elements E1 to E3 and the amount of light incident on the light receiving elements R1 to R4 from the radial artery 401 are suppressed even when a displacement occurs.
A description has been made of a fact that a decrease in the light amount according to a displacement can be suppressed by arrangement of the row of the light emitting elements E1 to E3 and the row of the light receiving elements R1 and R2 (or R3 and R4) in parallel with each other.
The arrangement according to the embodiment has been described in which the light emitting elements E1 to E3 and the light receiving elements R1 and R2 (or R3 and R4) are arranged in a staggered manner with the light emitting element E1 set at a left end and with the light emitting element E3 set at a right end. Arranging the row of the light emitting elements E1 to E3 and the row of the light receiving elements R1 and R2 (or R3 and R4) on the same straight line rather than in parallel with each other is not precluded. However, the parallel arrangement has an advantage that a distance between the light emitting elements E1 and E3 can be shortened while a mounting region for the light emitting elements and the light receiving elements is secured. This not only leads to a reduction in size of an arrangement region of the elements but also leads to maintaining a large amplitude of the pulse wave received by the light receiving elements.

A structure adopted in the case 2 will next be described.
According to the embodiment, in order to be able to detect the pulse wave with high accuracy, the photoelectric pulse wave sensor 100 has a function of changing the outputs of the light emitting elements E1 to E3.
Even when the photoelectric pulse wave sensor 100 is displaced from the normal position, the outputs of the light emitting elements E1 to E3 are changed in such a manner as to cancel out variation in the amplitude of the pulse wave caused by the displacement. Consequently, as indicated by the curve of the case 2 in FIG. 6, the amplitude of the pulse wave can be made constant irrespective of the displacement amount. At the time of detection of the pulse wave, the pulse wave flowing through the radial artery 401 cannot be reflected accurately when there is variation in the amplitude of the pulse wave. The accuracy of detection of the pulse wave is further improved by keeping the amplitude of the pulse wave constant with respect to the displacement amount.

A configuration of the photoelectric pulse wave sensor 100 will next be described.

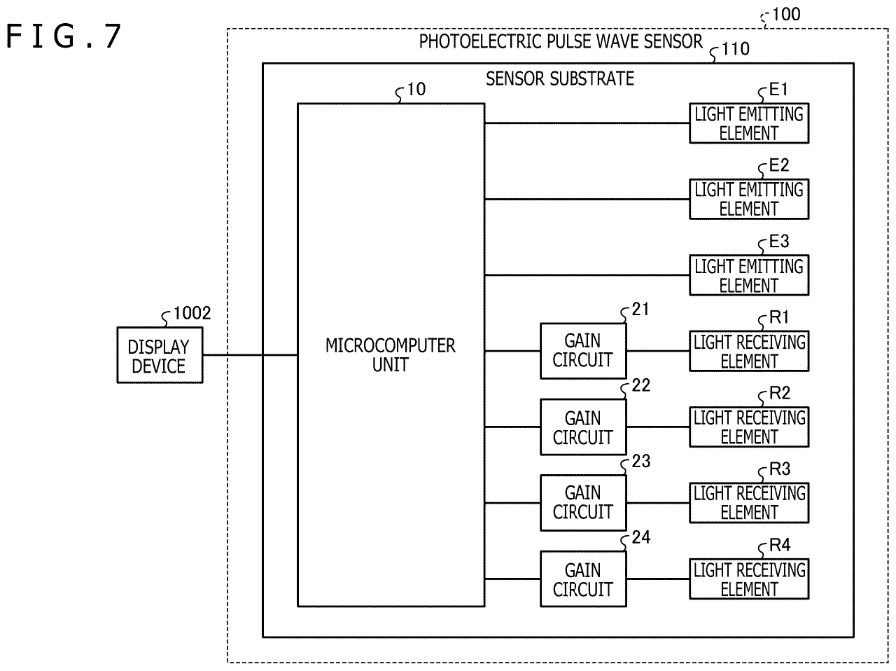

FIG. 7 is a schematic diagram illustrating an example of the configuration of the photoelectric pulse wave sensor 100.

The photoelectric pulse wave sensor 100 includes a microcomputer unit 10 and gain circuits 21 to 24 in addition to the sensor substrate 110, the light emitting elements E1 to E3, and the light receiving elements R1 to R4. Incidentally, the microcomputer unit 10 is an example of a processing circuit that adjusts the outputs of the light emitting elements.

The microcomputer unit 10 can supply electric power to the light emitting elements E1 to E3 and thus make the light emitting elements E1 to E3 emit light. The microcomputer unit 10 can independently adjust each of the output levels of the light emitting elements E1 to E3.

In addition, a signal corresponding to light received by the light receiving element R1 is amplified by the gain circuit 21 and is then input to the microcomputer unit 10.

A signal corresponding to light received by the light receiving element R2 is amplified by the gain circuit 22 and is then input to the microcomputer unit 10.

A signal corresponding to light received by the light receiving element R3 is amplified by the gain circuit 23 and is then input to the microcomputer unit 10.

A signal corresponding to light received by the light receiving element R4 is amplified by the gain circuit 24 and is then input to the microcomputer unit 10.

The microcomputer unit 10 makes the light emitting elements E1 to E3 emit light, receives the signals from the light receiving elements R1 to R4 via the gain circuits 21 to 24, and calculates the pulse wave on the basis of the received signals. The microcomputer unit 10 then outputs the pulse wave obtained by the calculation to the display device 1002.

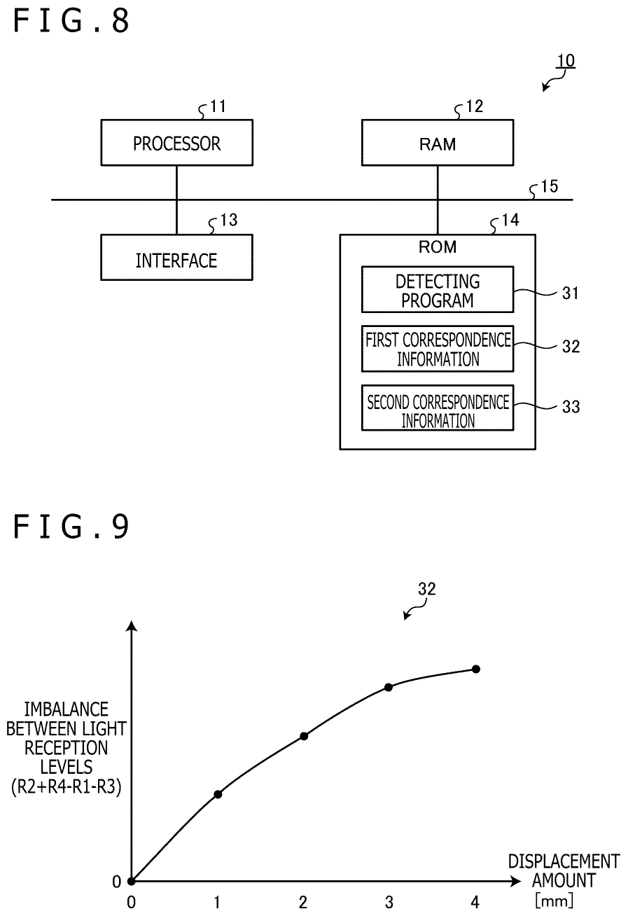

FIG. 8 is a schematic diagram illustrating an example of a configuration of the microcomputer unit 10.

The microcomputer unit 10 includes a processor 11, a random access memory (RAM) 12, an interface 13, a read only memory (ROM) 14, and a bus 15. The processor 11, the RAM 12, the interface 13, and the ROM 14 are electrically connected to the bus 15.

The interface 13 is a circuit for connection to an external device. The number and kind of the interface 13 included in the microcomputer unit 10 are not limited to one. In the example illustrated in FIG. 7, the interface 13 is connected with the light emitting elements E1 to E3, the gain circuits 21 to 24, and the display device 1002.

The processor 11 is a circuit that can execute a computer program. The processor 11 is a central processing unit (CPU), for example. The processor 11 collectively controls various constituent elements of the photoelectric pulse wave sensor 100 on the basis of a detecting program 31 stored in a predetermined position (ROM 14 as an example in this case) in advance and thus implements operation of the photoelectric pulse wave sensor 100.

Incidentally, a part or the whole of the operation of the photoelectric pulse wave sensor 100 (operation illustrated in FIG. 11 to be described later, for example) by the processor 11 described in the present specification may be implemented by a field-programmable gate array (FPGA), an application specific integrated circuit (ASIC), or a desired hardware circuit.

The RAM 12 is a volatile memory capable of high-speed operation, the volatile memory being used, for example, as an area into which the computer program is loaded, a data buffer, or a data cache. For example, the processor 11 loads the detecting program 31 into the RAM 12 from the ROM 14 and executes the detecting program 31 in the RAM 12.

The ROM 14 can store not only the detecting program 31 but also various kinds of data in advance. In the present example, the ROM 14 stores the detecting program 31, first correspondence information 32, and second correspondence information 33.

The processor 11 changes an output balance of the light emitting elements E1 to E3 according to the displacement amount in the circumferential direction of the arm 400 on the basis of the first correspondence information 32 and the second correspondence information 33.

FIG. 9 is a schematic diagram of assistance in explaining contents of the first correspondence information 32. In the present figure, an axis of abscissas indicates the displacement amount. An axis of ordinates indicates an imbalance in the Y-direction between light reception levels of the light receiving elements R1 to R4.
Here, as an example, the imbalance in the Y-direction between the light reception levels of the light receiving elements R1 to R4 is a value obtained by subtracting a total of the signal strength of the light receiving element R1 and the signal strength of the light receiving element R3 from a total of the signal strength of the light receiving element R2 and the signal strength of the light receiving element R4. A signal strength is, for example, the amplitude of the pulse wave output by the light receiving element.
That is,
Output Difference Df due to Displacement=(I(R2)+I(R4))−(I(R1)+I(R3))
I: Signal strength
As illustrated in FIG. 5, at the time of a normal arrangement, the light receiving elements R2 and R4 are arranged in a first region on a right side of the radial artery in a paper plane. The light receiving elements R1 and R3 are arranged in a second region on a left side of the radial artery in the paper plane in such a manner as to be equally separated from the radial artery.

In the case 1 in which the output levels of the light emitting elements E1 to E3 are fixed, when the photoelectric pulse wave sensor 100 is displaced in the circumferential direction of the arm 400, the imbalance between the light reception levels of the light receiving elements R1 to R4 changes according to the displacement amount.
For example, it can be read from FIG. 9 that the light reception levels of the light receiving elements R2 and R4 become higher than the light reception levels of the light receiving elements R1 and R3 when the photoelectric pulse wave sensor 100 is displaced in a downward direction from the normal position with respect to the paper plane of FIG. 5. It is understood that the imbalance (difference) according to the displacement amount is in a substantially linear relation.
With this displacement, the light emitting elements E1 and E2 move away from the artery, thus decreasing the amount of light entering the radial artery. In addition, the light emitting element E3 approaches the artery, thus increasing the amount of light entering the radial artery. Meanwhile, the output difference Df is large when the displacement amount is large, and the output difference Df is small when the displacement amount is small.
Hence, when Df in FIG. 9 is detected, the displacement amount can be computed. The light emission intensities of the light emitting elements E1, E2, and E3 can be adjusted according to the displacement amount.
A relation between the displacement amount of the photoelectric pulse wave sensor 100 and the imbalance in the Y-direction between the light reception levels of the light receiving elements R1 to R4 is obtained in advance under conditions where the light emitting elements E1 to E3 are driven to emit light at a predetermined output level. The relation is stored as the first correspondence information 32 in the ROM 14.

The processor 11 detects the imbalance Df in the Y-direction between the light reception levels of the light receiving elements R1 to R4 in predetermined timing, and calculates the displacement amount on the basis of the detected imbalance Df and the first correspondence information 32.

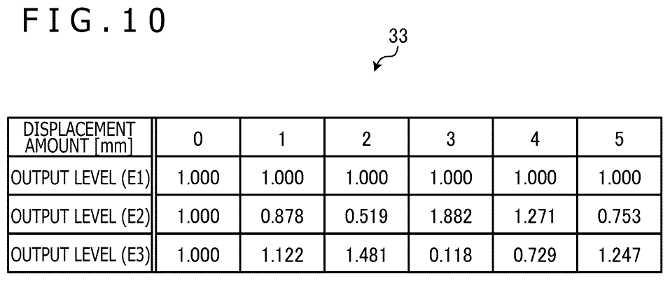

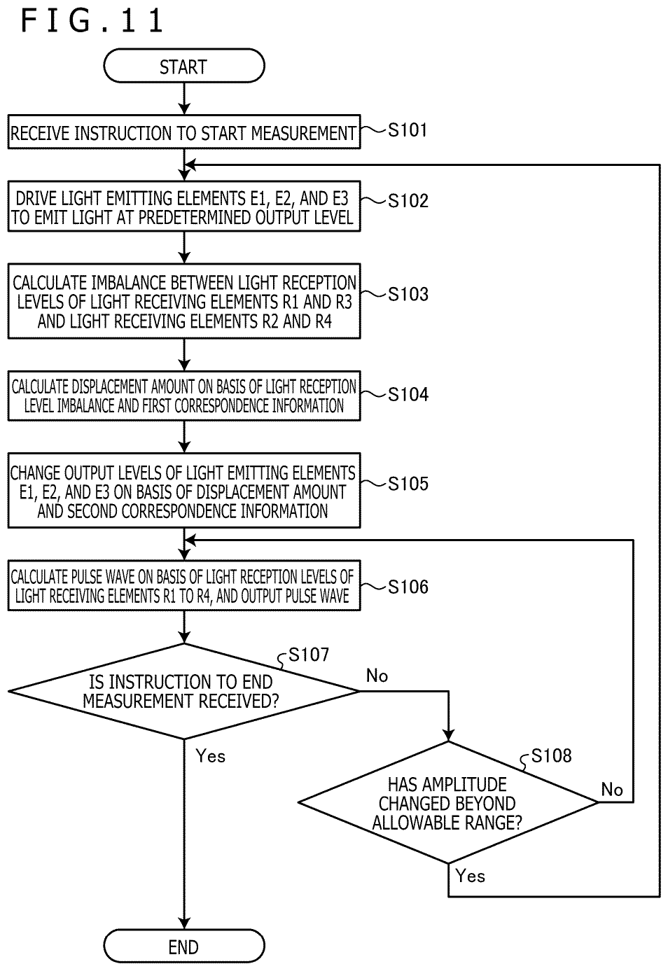

FIG. 10 is a diagram illustrating an example of contents of the second correspondence information 33.
The second correspondence information 33 has a table structure. However, the data structure of the second correspondence information 33 may be configured by an equation.

As illustrated in FIG. 10, the second correspondence information 33 represents a relation between the displacement amount and set values of the output levels of the light emitting elements E1 to E3. Changing the set values of the output levels of the light emitting elements E1 to E3 as illustrated in FIG. 10 can cancel out variation in the amplitude of the pulse wave when the photoelectric pulse wave sensor 100 is displaced from the normal position. For example, when the displacement amount is 1 mm, variation in the amplitude of the pulse wave is eliminated by not changing the output level of the light emitting element E1, lowering the output level of the light emitting element E2, and raising the output level of the light emitting element E3. For example, when the displacement amount is 3 mm, variation in the amplitude of the pulse wave is eliminated by not changing the output level of the light emitting element E1, raising the output level of the light emitting element E2, and lowering the output level of the light emitting element E3.
Variation in the amplitude of the pulse wave may be canceled out by changing all of the output levels of the light emitting elements E1 to E3. Variation in the amplitude of the pulse wave may be canceled out by only raising at least one of the output levels of the light emitting elements E1 to E3, or variation in the amplitude of the pulse wave may be canceled out by only lowering at least one of the output levels of the light emitting elements E1 to E3.
However, when at least one of the output levels of the light emitting elements E1 to E3 is raised and at least another one of the output levels of the light emitting elements E1 to E3 is lowered at the time of occurrence of a displacement as illustrated in FIG. 10, variation in the amplitude of the pulse wave can be canceled out without the output levels of the individual light emitting elements being greatly changed.
The set values of the output levels of the light emitting elements E1 to E3 at each of the displacement amounts are determined for each light emitting element E. The set values of the output levels of the light emitting elements E1 to E3 which set values correspond to the displacement amounts can be calculated by performing an optical simulation, for example.

The processor 11 obtains the set values of the output levels of the light emitting elements E1 to E3 which set values correspond to the calculated displacement amount, from the second correspondence information 33, and makes the light emitting elements E1 to E3 emit light at the obtained set values.

FIG. 11 is a flowchart illustrating an example of operation of the smart watch 1000.

Step 101: The processor 11 receives an instruction to start a measurement issued by an operation of the wearer or another trigger (S101).
Step 102: The processor 11 drives the light emitting elements E1, E2, and E3 to emit light at predetermined output levels (S102).
The predetermined output levels are output levels same as the output levels of the light emitting elements E1, E2, and E3 at the time of generation of the first correspondence information 32.

For example, in a case where the relation between the displacement amount of the photoelectric pulse wave sensor 100 and the imbalance in the Y-direction between the light reception levels of the light receiving elements R1 to R4, the relation being recorded as the first correspondence information 32, is obtained under conditions where the light emitting elements E1, E2, and E3 are all driven at an output level “1,” the light emitting elements E1, E2, and E3 are all driven at the output level “1” in S102. Incidentally, the predetermined output levels can be changed freely as long as the predetermined output levels are the same as the output levels of the light emitting elements E1, E2, and E3 at the time of generation of the first correspondence information 32. For example, only the light emitting element E2 may be driven at the output level “1,” and the light emitting elements E1 and E3 may be turned off.

Incidentally, the predetermined output levels in S102 are an example of first set outputs.

Step 103: Following S102, the processor 11 calculates an imbalance (difference) between the light reception levels of the light receiving elements R1 and R3 and the light reception levels of the light receiving elements R2 and R4 (S103).
In an example, the imbalance is obtained by subtracting the total of the signal strength of the light receiving element R1 and the signal strength of the light receiving element R3 from the total of the signal strength of the light receiving element R2 and the signal strength of the light receiving element R4.

Step 104: The processor 11 calculates the displacement amount on the basis of the imbalance between the light reception levels and the first correspondence information 32 (S104).
The processor 11 obtains the displacement amount corresponding to the calculated value of the imbalance between the light reception levels by referring to the first correspondence information 32.

Step 105: The processor 11 individually changes the output levels of the light emitting elements E1, E2, and E3 on the basis of the displacement amount and the second correspondence information 33 (S105).
The processor 11 obtains a set of set values of the output levels of the light emitting elements E1, E2, and E3 which set corresponds to the calculated value of the displacement amount, by referring to the second correspondence information 33, and individually changes the output levels of the light emitting elements E1, E2, and E3 according to the obtained set.

Step 106: The light emitted from the light emitting elements E1, E2, and E3 after the changing of the output levels enters the light receiving elements R1 to R4. The processor 11 detects the pulse wave on the basis of the light reception levels of the light receiving elements R1 to R4 after the changing of the output levels, and outputs the pulse wave to the display device 1002 (S106).

Step 107: The processor 11 determines whether or not an instruction to end the measurement issued by an operation of the wearer or another trigger has been received (S107).
Step 108: When the instruction to end the measurement has not been received (in the case of No in S107), the processor 11 determines whether or not the amplitude of the pulse wave has changed beyond a predetermined allowable range (S108).

When the amplitude of the pulse wave has not changed beyond the predetermined allowable range (in the case of No in S108), the control proceeds to S106, and the operation of detecting and outputting the pulse wave continues.

When the amplitude of the pulse wave has changed beyond the predetermined allowable range (in the case of Yes in S108), the control proceeds to S102, and individual changing of the output levels of the light emitting elements E1, E2, and E3 is performed.

When the instruction to end the measurement has been received (in the case of Yes in S107), the processor 11 ends the series of operating steps related to the detection of the pulse wave.

As described above, according to the embodiment, the plurality of light emitting elements E are arranged in one row on the principal surface of the sensor substrate 110, and a plurality of light receiving elements R are arranged in one row in such a manner as to be in parallel with the row of the plurality of light emitting elements E.
When the living body is irradiated with the light from the plurality of light emitting elements E, the microcomputer unit 10 changes the output levels of the plurality of light emitting elements E differently for each light emitting element E according to the imbalance (difference) between the plurality of light receiving elements R in the return light received from the living body by the plurality of light receiving elements R. Then, the microcomputer unit 10 detects the pulse wave of the living body on the basis of the return light received by the plurality of light receiving elements R after the changing of the light output levels.

Accordingly, the pulse wave can be detected with high accuracy even when the photoelectric pulse wave sensor 100 is displaced from the position that the radial artery 401 passes. Hence, the detection of the pulse wave can be implemented with high accuracy even when the wearer fastens the belt 1003 loosely.

Incidentally, according to the embodiment, the microcomputer unit 10 makes the plurality of light emitting elements E emit light at the predetermined output levels (that is, at the first set outputs), and changes the light output levels differently for each light emitting element E on the basis of the imbalance in the return light received from the living body by the plurality of light receiving elements R.

In addition, according to the embodiment, as is clear from the series of loop processing steps of S102 to S106, S107 (No), and S108 (No) in FIG. 11, the microcomputer unit 10 repeatedly performs making the plurality of light emitting elements E emit light at the first set outputs, changing the light output levels differently for each light emitting element E on the basis of the imbalance, and detecting the pulse wave after the changing of the light output levels.

Accordingly, even when the displacement amount of the photoelectric pulse wave sensor 100 in the circumferential direction of the arm 400 is changed by body motion of the wearer or other causes, the light output levels can be changed dynamically according to the displacement amount.

Incidentally, in the above description, a difference between the total of the signal strength of the light receiving element R2 and the signal strength of the light receiving element R4 and the total of the signal strength of the light receiving element R1 and the signal strength of the light receiving element R3 is calculated as the imbalance between the light reception levels. An example of the imbalance between the light reception levels is not limited to this. A difference between the reception strengths of at least two of the plurality of light receiving elements R arranged in parallel with the row of the light emitting elements E1 to E3 can be used as the imbalance between the light reception levels. Hence, for example, a difference between the signal strength of the light receiving element R1 and the signal strength of the light receiving element R2 and a difference between the signal strength of the light receiving element R3 and the signal strength of the light receiving element R4 can be used as the imbalance between the light reception levels. In a case where three or more light receiving elements R are arranged in parallel with the row of the light emitting elements E1 to E3, a difference between the signal strengths of two light receiving elements R among the three or more light receiving elements R can be used as the imbalance between the light reception levels.

In addition, according to the embodiment, the smart watch 1000 as the pulse wave detecting device includes the belt 1003 for fitting the pulse wave detecting device to the arm with the principal surface of the sensor substrate 110 directed to the skin of the arm 400. The plurality of light emitting elements E and the plurality of light receiving elements R are both arranged in one row in a direction intersecting the direction in which the radial artery 401 of the arm extends.

Accordingly, even when the photoelectric pulse wave sensor 100 is displaced in the circumferential direction of the arm 400, the pulse wave can be detected with high accuracy.

In addition, according to the embodiment, in a case where the sensor substrate 110 is displaced from the normal position on the arm 400 in a direction intersecting the direction in which the radial artery 401 extends, the microcomputer unit 10 changes the light output levels differently for each light emitting element in such a manner as to cancel variation in the amplitude of the pulse wave caused by the displacement of the sensor substrate 110 from the normal position.

Even when the photoelectric pulse wave sensor 100 is displaced in the circumferential direction of the arm 400, variation in the amplitude of the pulse wave according to the displacement can be suppressed.

In addition, according to the embodiment, the microcomputer unit 10 calculates the displacement amount on the basis of the imbalance between the light reception levels, and changes the light outputs differently for each light emitting element E on the basis of the calculated displacement amount. The microcomputer unit 10 does not necessarily have to calculate the displacement amount. Correspondence information obtained by recording a relation between imbalances in the return light and sets of set values of the output levels of the plurality of light emitting elements E may be stored in the ROM 14 in advance, and the microcomputer unit 10 may calculate a set of set values of the output levels of the plurality of light emitting elements E on the basis of the calculated imbalance in the return light and the correspondence information.

Modes of the pulse wave detecting device according to the embodiment are as described in the following, for example.

### Note 1

A pulse wave detecting device includes:
a sensor substrate;
a plurality of light emitting elements arranged in a first row on a principal surface of the sensor substrate and configured to emit light;
a plurality of light receiving elements arranged in a second row parallel with the first row on the principal surface and configured to receive return light,
one or more light receiving elements of the plurality of light receiving elements being provided in a first region of the principal surface, one or more light receiving elements of the plurality of light receiving elements being provided in a second region of the principal surface, the principal surface being divided into the first region and the second region, the first region and the second region having therebetween a boundary orthogonal to a direction of the second row; and
a processing circuit configured to change light outputs of the plurality of light emitting elements differently for each light emitting element on the basis of a first signal output by the one or more light receiving elements provided in the first region and a second signal output by the one or more light receiving elements provided in the second region, and detect a pulse wave on the basis of signals output by the plurality of light receiving elements after the changing of the light outputs.

### Note 2

In the pulse wave detecting device according to note 1,
the processing circuit changes the light outputs differently for each light emitting element on the basis of the first signal and the second signal output by the plurality of light receiving elements when the plurality of light emitting elements are made to emit the light at a fixed output.

### Note 3

In the pulse wave detecting device according to note 1,
the processing circuit changes the light outputs differently for each light emitting element on the basis of a difference between the first signal and the second signal.

### Note 4

The pulse wave detecting device according to any one of note 1 to note 3 is
a wearable pulse wave detecting device further including a fitting belt.

Incidentally, in the embodiment, a description has been made of the smart watch 1000 as an example of the wearable pulse wave detecting device.

### Note 5

In the pulse wave detecting device according to any one of note 1 to note 3,
the plurality of light emitting elements and the plurality of light receiving elements are disposed in a staggered manner.

### Note 6

In the pulse wave detecting device according to any one of note 1 to note 3,
the processing circuit increases the light output of at least one light emitting element of the plurality of light emitting elements and increases the light output of at least another light emitting element of the plurality of light emitting elements.

### Note 7

A wearable pulse wave detecting device is
a wearable pulse wave detecting device having a pulse wave detecting function, the wearable pulse wave detecting device including:
a substrate module including
   a sensor substrate,
   a central light emitting element mounted on the sensor substrate and provided at a center of the sensor substrate,
   a near side light emitting element and a far side light emitting element arranged on a near side and a far side at equal intervals with the central light emitting element at the center in such a manner as to form a first row,
   a first light receiving element and a third light receiving element provided such that an imaginary line connecting arrangement points of the first light receiving element and the third light receiving element intersects the first row and passes between the far side light emitting element and the central light emitting element, and
   a second light receiving element and a fourth light receiving element provided such that an imaginary line connecting arrangement points of the second light receiving element and the fourth light receiving element intersects the first row and passes between the near side light emitting element and the central light emitting element, the arrangement points of the first light receiving element and the third light receiving element and the arrangement points of the second light receiving element and the fourth light receiving element forming a rectangle;
a casing provided in such an orientation that the light emitting elements of the substrate module abut against a skin; and
a belt provided to both side surfaces of the casing.

### Note 8

In the wearable pulse wave detecting device according to note 7,
a second row connecting the arrangement points of the first light receiving element and the second light receiving element and a third row connecting the arrangement points of the third light receiving element and the fourth light receiving element are arranged to be separated from the first row at equal intervals.

### Note 9

In the wearable pulse wave detecting device according to note 8,
directions of the first row and a radial artery of an arm substantially coincide with each other, and
the wearable pulse wave detecting device includes a computing unit configured to
   compute a difference between a sum total output of the first light receiving element and the second light receiving element and a sum total output of the third light receiving element and the fourth light receiving element from the sum total outputs, and
   adjust outputs of the three light emitting elements.

The present invention produces an effect of being able to provide a pulse wave detecting device and a wearable pulse wave detecting device that can detect the pulse wave with high accuracy even when the sensor is displaced from the position that the blood vessel passes.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A pulse wave detecting device comprising:
a sensor substrate;
a plurality of light emitting elements arranged in one row on a principal surface of the sensor substrate and configured to emit light;
a plurality of light receiving elements arranged in one row parallel with the row of the plurality of light emitting elements on the principal surface and configured to receive return light; and
a processing circuit configured to change light outputs of the plurality of light emitting elements differently for each light emitting element on a basis of first signals output by at least two light receiving elements of the plurality of light receiving elements, and detect a pulse wave on a basis of second signals output by the plurality of light receiving elements after the changing of the light outputs.

2. The pulse wave detecting device according to claim 1, wherein,
when the pulse wave detecting device is fitted to an arm of a user, the processing circuit calculates a displacement amount of the plurality of light emitting elements in a circumferential direction of the arm from a position of the plurality of light emitting elements in a predetermined positional relation obtained with respect to a blood vessel of the arm, on a basis of a change in the first signal in each light receiving element, and changes the light outputs on a basis of the displacement amount.

3. The pulse wave detecting device according to claim 1, wherein
the processing circuit changes the light outputs differently for each light emitting element on the basis of the first signals output by the at least two light receiving elements when the plurality of light emitting elements are made to emit the light at a fixed output.

4. The pulse wave detecting device according to claim 1, wherein
the processing circuit changes the light outputs differently for each light emitting element on a basis of a difference between the first signals output by the at least two light receiving elements.

5. The pulse wave detecting device according to claim 1, further comprising:
a fitting belt.

6. The pulse wave detecting device according to claim 1, wherein
the plurality of light emitting elements and the plurality of light receiving elements are disposed in a staggered manner.

7. The pulse wave detecting device according to claim 1, wherein
the processing circuit increases the light output of at least one light emitting element of the plurality of light emitting elements and decreases the light output of at least another light emitting element of the plurality of light emitting elements.
